(19) United States
(12) Patent Application Publication
(10) Pub. No.: US 2023/0338671 A1
(43) Pub. Date: Oct. 26, 2023

(54) Injector Device

(71) Applicant: Sanofi, Paris (FR)

(72) Inventors: Uwe Dasbach, Frankfurt am Main (DE); Kai Scheinert, Frankfurt am Main (DE); Daniel Auernhammer, Frankfurt am Main (DE); Florian Schauderna, Frankfurt am Main (DE); Thomas Mark Kemp, Herts Cambridgeshire (GB); Tim Schuller, Herts Cambridgeshire (GB); Robbie Wilson, Herts Cambridgeshire (GB); Michael Noble, Herts Cambridgeshire (GB); Ryan Anthony McGinley, Herts Cambridgeshire (GB)

(21) Appl. No.: 17/916,144

(22) PCT Filed: Mar. 31, 2021
(86) PCT No.: PCT/EP2021/058499

§ 371 Date: Sep. 30, 2022

(30) Foreign Application Priority Data
Apr. 3, 2020 (EP) 20315112.1

(51) Int. Cl. A61M 5/44; A61M 5/32

(52) U.S. Cl. CPC A61M 5/445; A61M 5/3202; A61M 5/3232; A61M 5/3243; A61M 2205/584

(57) ABSTRACT

A drug delivery device comprises an outer surface and a region for receiving a medicament container. The region has an inner surface to contact and support a medicament container received therein. The device also includes a thermal bridge extending between the inner surface and the outer surface to conduct heat between the inner surface and the outer surface. The thermal bridge has a thermal conductivity equal to or greater than 10W/mk.

### CROSS REFERENCE TO RELATED APPLICATIONS

The present application is the national stage entry of International Patent Application No. PCT/EP2021/058499, filed on Mar. 31, 2021, and claims priority to Application No. EP 20315112.1, filed on Apr. 3, 2020, the disclosures of which are incorporated herein by reference.

### TECHNICAL FIELD

The present disclosure relates to an injector device for a medicament.

### BACKGROUND

Injection devices, for example auto-injectors, typically have a sealed container of medicament, and a needle for injection of the medicament into a patient. During injection, a drive mechanism displaces a plunger into the medicament container to dispense medicament through the needle.

It is often necessary to store the injection device in a refrigerator to keep the medicament at a suitable storage temperature. The storage temperature will be significantly less than body temperature and this can cause some discomfort for patients during injection.

### SUMMARY

According to the present disclosure, there is provided a drug delivery device comprising an injector device comprising: an outer surface; a region for receiving a medicament container, the region having at least one inner surface to contact and support a medicament container received therein; and a thermal bridge extending between the at least one inner surface and the outer surface to conduct heat between the inner and outer surfaces, wherein the thermal bridge has a thermal conductivity equal to or greater than 10 W/mk.

Therefore, the device is configured to allow the medicament to warm up to more suitable injection temperature quicker after removal from a refrigerator.

The inner and outer surfaces may be integrally formed with the thermal bridge to provide an unbroken conductive pathway between the inner and outer surfaces.

Therefore heat transfer between the inner and outer surfaces is maximised.

Said outer surface may be an outer surface of the housing.

Therefore the medicament is more efficiently heated as the device is handled, the outer surface transferring heat away from the user’s hands.

The thermal bridge may be integrally formed as part of the housing.

Therefore the device is simply constructed.

The thermal bridge may comprise a region of the housing having increased wall thickness so that an internal diameter of said region of increased wall thickness is substantially the same as an external diameter of a medicament container to be received therein.

Therefore the thermal bridge extends all the way around the medicament container to maximise the area of contact between the medicament container and the thermal bridge for heat transfer.

The housing may comprise first and second parts, and wherein the thermal bridge is integrally formed in the first part.

Therefore only the first part of the housing need comprise a material having a thermal conductivity greater than 10 W/mk which makes the device cheaper to manufacture.

The thermal bridge may comprise a collar disposed between first and second parts of the housing, wherein the collar comprises a cylindrical section with an internal diameter substantially the same as an external diameter of a medicament container to be received therein.

Therefore only the collar need comprise a material having a thermal conductivity greater than 10W/mk which makes the device cheaper to manufacture. Further, as the first and second parts of the housing may be made from conventional plastic materials - which are not as cold to the touch as more thermally conductive materials - the device is more comfortable to handle when removed from a refrigerator.

The thermal bridge may comprise a material having a coefficient of thermal conductivity greater than 70W/mK.

Therefore heat is even more quickly transferred between the inner and outer surfaces.

The thermal bridge may extend into contact with 20% of the outer surface of the medicament container.

Therefore a large contact area is provided between the medicament container and the thermal bridge for efficient heat transfer.

The thermal bridge may hold the medicament container in a fixed position relative to the housing by interference fit.

The thermal bridge may hold the medicament container in a fixed position relative to the housing, the thermal bridge being bonded to the medicament container by an adhesive having a thermal conductivity greater than 2 W/mK.

The medicament container may comprise a material having a coefficient of thermal conductivity greater than 10 W/mK.

The thermal bridge may comprise a thermochromic material configured to change color in dependence on the temperature of the thermal bridge.

Therefore, a user can see if the device has warmed up.

The outer surface of the device may comprise a colored region which corresponds to the color of the thermochromic material when the thermal bridge reaches a predetermined temperature.

Therefore, a user can see if the device has warmed up to an extent that the medicament has reached a comfortable injection temperature.

Also according to the present disclosure, there is provided a system, comprising a heater and the device according to any of claims 1 to 14, wherein the heater comprises a U-shaped channel for receiving the device so that a heated surface of the U-shaped channel contacts the outer surface of the device when the device is received therein.

Also according to the present disclosure, there is provided a method of operating a drug delivery device, the drug delivery comprising:
an outer surface;
a medicament container, and
a thermal bridge extending between the medicament container and the outer surface, the thermal bridge having a thermal conductivity equal to or greater than 10W/mk;
wherein the method comprises
   placing the outer surface of the drug delivery device in contact with a heat source to heat medicament within the medicament container from a storage temperature to an injection temperature.

The heater allows the device to be brought up to a comfortable injection temperature more quickly.

### BRIEF DESCRIPTION OF THE FIGURES

Embodiments of the present disclosure will now be described, by way of example only, with reference to the accompanying drawings, in which:

FIG. 1A is a schematic side view of an injector device that embodies the present disclosure, and a removable cap;

FIG. 1B is a schematic side view of the injector device of FIG. 1A, with the cap removed from the housing;

FIG. 2 is cross-section of one embodiment of the present disclosure;

FIG. 3 is cross-section of another embodiment of the present disclosure;

FIG. 4 is cross-section of another embodiment of the present disclosure;

FIG. 5 is cross-section of another embodiment of the present disclosure;

FIG. 6 shows a system in accordance with the teachings of the present disclosure.

### DETAILED DESCRIPTION

A drug delivery device, as described herein, may be configured to inject a medicament into a patient. For example, delivery could be sub-cutaneous, intra-muscular, or intravenous. Such a device could be operated by a patient or care-giver, such as a nurse or physician, and can include various types of safety syringe, pen-injector, or auto-injector. The device can include a cartridge-based system that requires piercing a sealed ampule before use. Volumes of medicament delivered with these various devices can range from about 0.5 ml to about 3 ml. Another device may comprise a pre-filled syringe within a housing of the device. The syringe may be fixed within the housing or may be moveable within the housing, for example from a retracted position to an operation extended position.

In combination with a specific medicament, the presently described devices may also be customized in order to operate within required specifications. For example, the device may be customized to inject a medicament within a certain time period (e.g., about 3 to about 20 seconds for auto-injectors). Other specifications can include a low or minimal level of discomfort, or to certain conditions related to human factors, shelf-life, expiry, biocompatibility, environmental considerations, etc. Such variations can arise due to various factors, such as, for example, a drug ranging in viscosity from about 3 cP to about 50 cP. Consequently, a drug delivery device will often include a hollow needle ranging from about 25 to about 31 Gauge in size. Common sizes are 17 and 29 Gauge.

The delivery devices described herein can also include one or more automated functions. For example, one or more of combining the needle and cartridge, needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device.

The one or more automated functions of an auto-injector may each be activated via an activation mechanism. Such an activation mechanism can include an actuator, for example, one or more of a button, a lever, a needle sleeve, or other activation component. Activation of an automated function may be a one-step or multi-step process. That is, a user may need to activate one or more activation components in order to cause the automated function. For example, in a one-step process, a user may depress a needle sleeve against their body in order to cause injection of a medicament. Other devices may require a multi-step activation of an automated function. For example, a user may be required to depress a button and retract a needle shield in order to cause injection.

In addition, activation of one automated function may activate one or more subsequent automated functions, thereby forming an activation sequence. For example, activation of a first automated function may activate at least two of combining the needle and cartridge, needle insertion, medicament injection, and needle retraction. Some devices may also require a specific sequence of steps to cause the one or more automated functions to occur. Other devices may operate with a sequence of independent steps.

Some delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

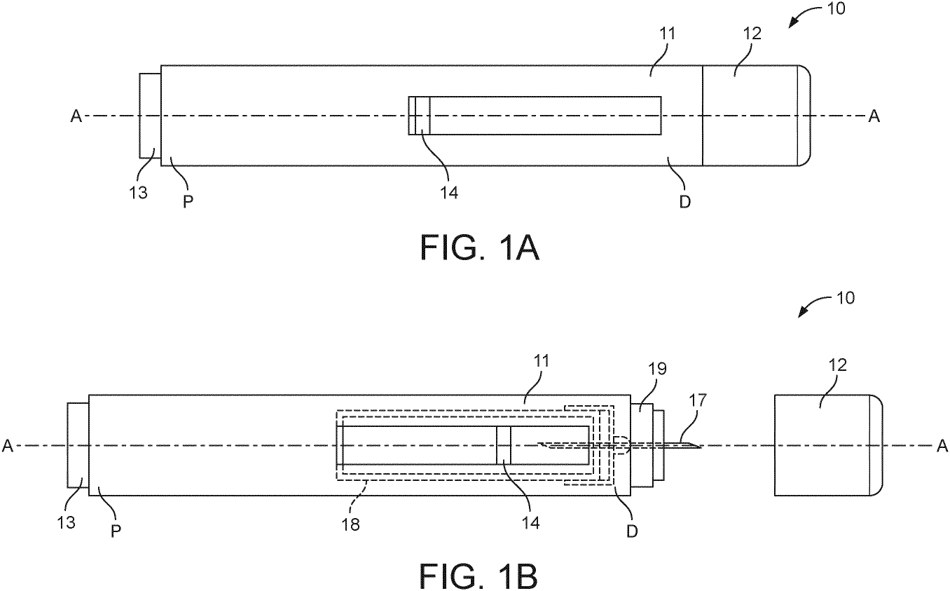

According to some embodiments of the present disclosure, an exemplary drug delivery device 10 is shown in FIGS. 1A and 1B. Device 10, as described above, is configured to inject a medicament into a patient’s body. Device 10 includes a housing 11 which contains a medicament container 18 that defines a reservoir containing the medicament to be injected, and the components required to facilitate one or more steps of the delivery process. The medicament container 18 may be a cartridge or pre-filled syringe.

The device 10 can also include a cap 12 that can be detachably mounted to the housing 11. Typically, a user must remove cap 12 from housing 11 before device 10 can be operated.

As shown, housing 11 is substantially cylindrical and has a substantially constant diameter along the longitudinal axis A-A. The housing 11 has a distal region D and a proximal region P. The term “distal” refers to a location that is relatively closer to a site of injection, and the term “proximal” refers to a location that is relatively further away from the injection site.

Device 10 can also include a needle sleeve 19 coupled to housing 11 to permit movement of sleeve 19 relative to housing 11. For example, sleeve 19 can move in a longitudinal direction parallel to longitudinal axis A-A. Specifically, movement of sleeve 19 in a proximal direction can permit a needle 17 to extend from distal region D of housing 11.

Insertion of needle 17 can occur via several mechanisms. For example, needle 17 may be fixedly located relative to housing 11 and initially be located within an extended needle sleeve 19. Proximal movement of sleeve 19 by placing a distal end of sleeve 19 against a patient’s body and moving housing 11 in a distal direction will uncover the distal end of needle 17. Such relative movement allows the distal end of needle 17 to extend into the patient’s body. Such insertion is termed “manual” insertion as needle 17 is manually inserted via the patient’s manual movement of housing 11 relative to sleeve 19.

Another form of insertion is “automated”, whereby needle 17 moves relative to housing 11. Such insertion can be triggered by movement of sleeve 19 or by another form of activation, such as, for example, a button 13. As shown in FIGS. 1A and 1B, button 13 is located at a proximal end of housing 11. However, in other embodiments, button 13 could be located on a side of housing 11.

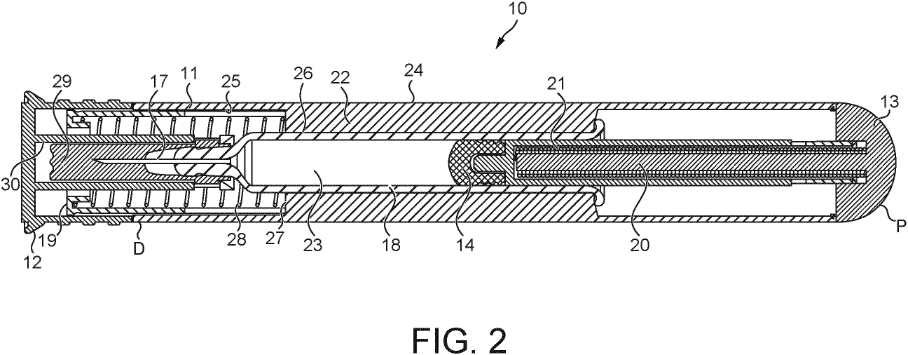

Other manual or automated features can include drug injection or needle retraction, or both. Injection is the process by which a bung or piston 14 is moved from a proximal location to a more distal location within the reservoir of the cartridge 18 in order to force a medicament from the cartridge 18 through needle 17. In some embodiments, a drive mechanism 20 (as shown in FIGS. 2 to 5) is activated to force medicament from the cartridge 18. The drive mechanism 20 comprises a drive spring 21. The drive spring 21 is under compression before drive mechanism 20 is activated. A proximal end of the drive spring 21 can be fixed within proximal region P of housing 11, and a distal end of the drive spring 21 can be configured to apply a compressive force to a proximal surface of piston 14. Following activation, at least part of the energy stored in the drive spring 21 can be applied to the proximal surface of piston 14. This compressive force can act on piston 14 to move it in a distal direction. Such distal movement acts to compress the liquid medicament within the cartridge 18, forcing it out of needle 17.

Following injection, needle 17 can be retracted within sleeve 19 or housing 11. Retraction can occur when sleeve 19 moves distally as a user removes device 10 from a patient’s body. This can occur as needle 17 remains fixedly located relative to housing 11. Once a distal end of sleeve 19 has moved past a distal end of needle 17, and needle 17 is covered, sleeve 19 can be locked. Such locking can include locking any proximal movement of sleeve 19 relative to housing 11.

Another form of needle retraction can occur if needle 17 is moved relative to housing 11. Such movement can occur if the cartridge 18 within housing 11 is moved in a proximal direction relative to housing 11. This proximal movement can be achieved by using a retraction spring (not shown), located in distal region D. A compressed retraction spring, when activated, can supply sufficient force to the cartridge 18 to move it in a proximal direction. Following sufficient retraction, any relative movement between needle 17 and housing 11 can be locked with a locking mechanism. In addition, button 13 or other components of device 10 can be locked as required.

FIG. 2 shows an injector device 10 of one embodiment of the present disclosure, in which like features retain the same reference numbers.

The device 10 comprises a thermal bridge 22 configured to conduct heat between the medicament cartridge 18 and an outer surface 24 of the device 10. Therefore, heat applied to the outer surface 24 of the device 10 is conducted to the medicament cartridge 18 to heat medicament retained therein.

In use, the device 10 may be stored in a refrigerator to keep the medicament at a storage temperature. The storage temperature will be significantly less than body temperature. Storage temperatures vary, but are usually between -4 and 4° C.

A patient’s perception of pain during an injection is known to correspond to the temperature of the medicament injected. Medicaments injected at their storage temperature can cause an increased perception of pain relative to medicaments injected at warmer temperatures. Ideally, the medicament is about body temperature during injection to minimise pain perception.

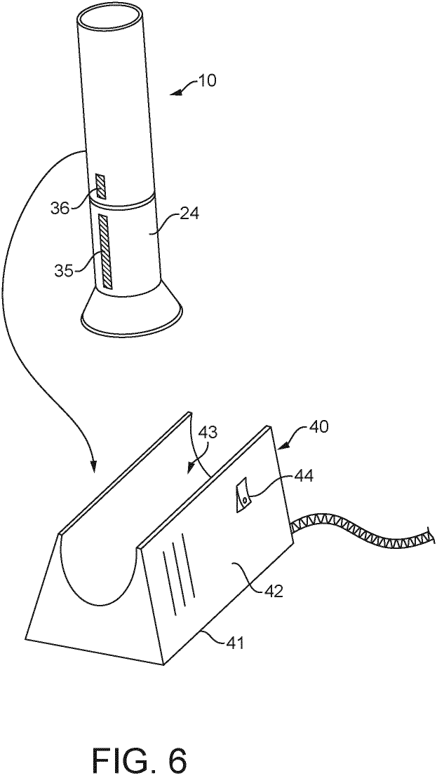

Therefore, by providing a thermal bridge 22 between the outer surface 24 of the device 10 and the medicament cartridge 18, it is possible to more quickly heat the medicament by applying heat to the outer surface 24. Where the ambient temperature is greater than the storage temperature, heat may be applied passively simply by leaving the device 10 out of the refrigerator. Alternatively, heat may be applied by a user’s hands as the user grips the device 10. It is also envisaged that a dock 40 may be provided to actively heat the device, as explained further below with reference to FIG. 6.

In the embodiment illustrated by FIG. 2, the thermal bridge 22 is integrally formed with the housing 11 and upstands from an inner surface 25 of the housing 11 and into contact with an outer surface 26 of the medicament cartridge 18. FIG. 2 shows a section taken through the device 10. Because the thermal bridge 22 is integrally formed with the housing 11, the thermal bridge 22 provides an unbroken conductive pathway between the outer surface 26 of the medicament cartridge 18 and the outer surface 24 of the housing 11.

In the illustrated embodiment, the thermal bridge 22 comprises a region of the housing 11 having increased wall thickness so that the internal diameter of said region is reduced relative to the rest of the housing 11. The internal diameter is substantially the same as the external diameter of the medicament cartridge 18 so that the inner surface 25 of said region tightly abuts the outer surface 26 of the medicament cartridge 18. Therefore, the thermal bridge 22 extends all the way around the circumference of the cartridge 18 to ensure significant contact area between the thermal bridge 22 and the cartridge 18. This provides the largest possible area for heat transfer, the importance of which the skilled person will readily appreciate from Fourier’s Law.

Advantageously, the thermal bridge 22 provides a shoulder 27 for supporting a proximal end of a sleeve retraction spring 28. The shoulder 27 is provided where the internal diameter of the housing 11 is stepped in to form the thermal bridge 22. The sleeve retraction spring 28 biases the sleeve 19 out of the distal end of the housing 11 to conceal the needle 17.

The cap 12 locates over the sleeve 19. A needle shield 29 is also provided to enclose the needle 17 for additional safety. The needle shield 29 is gripped by a wall 30 that extends from the cap 12 so that the needle shield 29 is removed simultaneously with the cap 12.

To maximise the heat transfer between the outer surface 24 of the housing 11 and the outer surface 26 of the cartridge 18, the thermal bridge 22 may be made from a material with a relatively high coefficient of thermal conductivity. By relatively high, it is meant relative to the usual family of plastics used to make housings for injector devices. In embodiments described herein the thermal bridge 22 has a coefficient of thermal conductivity greater than 10W/mK.

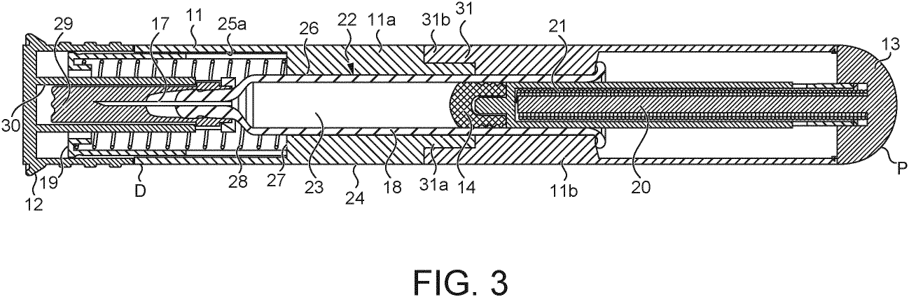

In another embodiment illustrated in FIG. 3 (like features retaining the same reference numbers) the housing 11 is split into first and second parts 11a and 11b. The first part 11a forms the distal region D of the device 10 and houses the medicament cartridge 18, while the second part 11b forms the proximal region P of the device 10 and houses the drive mechanism 20.

In this embodiment, the thermal bridge 22 is integrally formed with the first part 11a of the housing 11 and upstands from an inner surface 25a of the first part 11a of the housing 11 and into contact with an outer surface 26 of the medicament cartridge 18. FIG. 3 shows a section taken through the device 10. As in the embodiment of FIG. 2, because the thermal bridge 22 is integrally formed with the housing 11, the thermal bridge 22 provides an unbroken conductive pathway between the outer surface 26 of the medicament cartridge 18 and the outer surface 24 of the housing 11.

The thermal bridge 22 comprises a region of the first part 11a of the housing 11 having increased wall thickness so that the internal diameter of said region is reduced relative to the rest of the housing 11. As in the embodiment of FIG. 2, the internal diameter is substantially the same as the external diameter of the medicament cartridge 18 so that inner surface 25a of said region tightly abuts the outer surface 26 of the medicament cartridge 18.

The first part 11a of the housing 11 may attach to the second part 11b of the housing 11 by a threaded connection 31. In the illustrated embodiment, the first part 11a of the housing 11 comprises a male threaded end 31a which cooperates with a female threaded end 31b of the second part 11b of the housing 11 to attach the first and second parts 11a, 11b of the housing 11 together.

An advantage of splitting the first and second parts 11a, 11b of the housing 11 is that the first part 11a of the housing 11 and, therefore, the thermal bridge 22 may be made from a different, more thermally conductive material than the second part 11b of the housing 11, such as material having a thermal conductivity greater than 10 W/mK. Therefore, the cost of the device 10 may be kept in check where the more thermally conductive material has a higher unit cost.

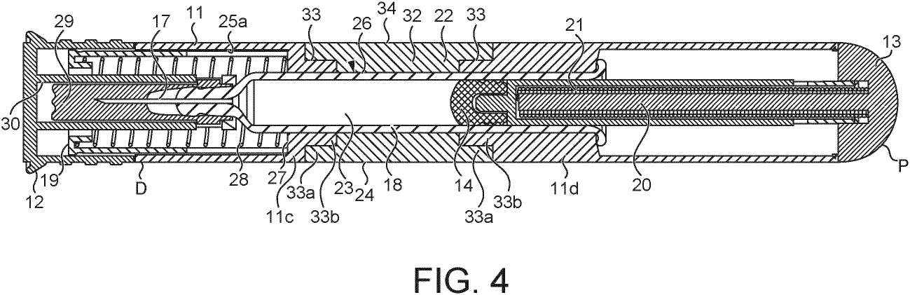

In another embodiment of the present disclosure, illustrated in FIG. 4 (like features retaining the same reference numbers), the housing 11 comprises first and second parts 11c, 11d and a collar 32 which separates the first and second parts 11c, 11d. The first part 11c of the housing 11 forms the distal end D of the device 10 and the second part 11d of the housing 11 forms the proximal end P of the device 10. In the embodiment of FIG. 4, the first and second parts 11c, 11d are not directly attached, but are each attached by a threaded connection 33 to opposite ends of the collar 32. The collar 32 is disposed over the cartridge 18. The collar 32 comprises a cylindrical section with an internal diameter substantially the same as the external diameter of the medicament cartridge 18 so that inner surface of the collar 32 tightly abuts the outer surface of the medicament cartridge 18. An outer surface 34 of the collar 32 is collinear with outer surfaces of the first and second parts 11c, 11d of the housing 11 and forms part of the outer surface 24 of the device 10. Therefore, the collar 32 provides the thermal bridge 22 and an unbroken conductive pathway between the outer surface 26 of the medicament cartridge 18 and the outer surface 24 of the device 10.

In the illustrated embodiment, each end of the collar 32 is provided with a female thread 33a to cooperate with corresponding male threaded ends 33b of the first and second parts 11c, 11d of the housing 11. An advantage of this embodiment is that the collar 32 and, therefore, the thermal bridge 22 may be made from a different, more thermally conductive material than the first and second parts 11c, 11d of the housing 11, such as material having a thermal conductivity greater than 10 W/mK. Therefore, the cost of the device 10 may be kept in check where the more thermally conductive material has a higher unit cost. Another advantage is that the first and second parts 11c, 11d of the housing 11 may be made from conventional plastic materials which are not as cold to the touch as more thermally conductive materials, making the device 10 more comfortable to handle when removed from a refrigerator.

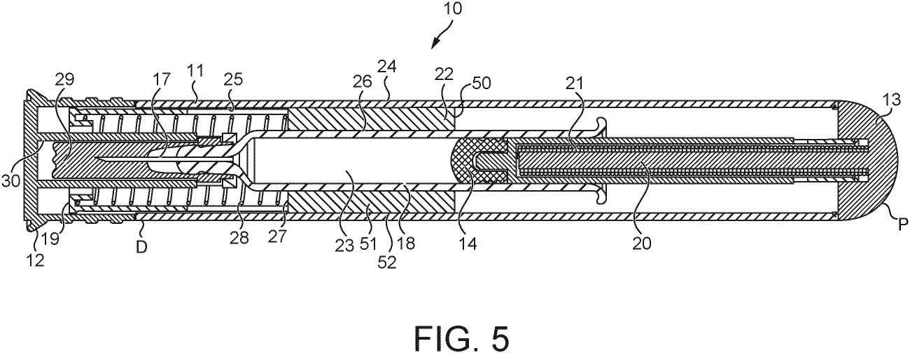

In another embodiment of the present disclosure illustrated in FIG. 5 (like features retaining the same reference numbers), the thermal bridge 22 comprises an insert 50 which is disposed between the housing 11 and the medicament cartridge 18. An inner surface 51 of the insert 50 locates against the outer surface 26 of the medicament cartridge 18, while an outer surface 52 of the insert 50 locates against the inner surface 25 of the housing 11. Therefore, the insert 50 provides a conductive pathway between the medicament cartridge 18 and the housing 11. The insert 50 is cylindrical so that the insert extends the whole way around the cartridge 18 to maximise the surface area in contact with the cartridge 18. However, the insert 50 may instead be split into individual bridging portions to save on material costs.

The insert 50 may hold the cartridge 18 in fixed relation with respect to the housing 11 by an interference fit between inner and outer surfaces 51, 52 of the insert 50 and corresponding surfaces 26, 25 of the cartridge 18 and housing 11, respectively. Alternatively, a thermally conductive adhesive may be used to bond inner and outer surfaces 51, 52 of the insert 50 to corresponding surfaces 26, 25 of the cartridge 18 and housing 11, respectively.

In the above described embodiments the thermal bridge 22 extends all the way around the circumference of the cartridge 18 to ensure significant contact area between the thermal bridge 22 and the cartridge 18. As discussed, this provides the largest possible area for heat transfer. The thermal bridge 22 extends along the length of the cartridge 18 so that the thermal bridge 22 is in contact with at least 50% of a medicament containing part 23 of the cartridge 18.

The thermal bridge 22 is configured to hold the medicament cartridge 18 in fixed relation relative to the housing 11. The thermal bridge 22 may hold the medicament container 18 by friction. For example, the medicament cartridge 18 may be held within the thermal bridge 22 by an interference fit. Alternatively an adhesive may be used between the thermal bridge 22 and the outer surface 26 of the medicament cartridge 18. Adhesives with a relatively high thermal conductivity can be selected, such a thermal conductivity greater than 2 W/mK.

Because an objective of the present disclosure is to increase the rate at which the medicament is brought up to a suitable temperature, the medicament cartridge 18 itself may also be made of a relatively more conductive material. By relatively more conductive, it is meant relative to convention materials for making cartridges, such as glass or the usual family of polymers. For example, the cartridge 18 may be made from stainless steel.

Also in embodiments of the present disclosure, a region of the outer surface 24 of the device 10 may be configured to change color in response to a change in temperature. For example, the outer surface 24 of the device 10 may be provided with a strip of thermochromic material 35 (as shown in FIG. 6). Therefore, as the device 10 warms up, the color of the outer surface 24 changes to signify this change in temperature to a user. The outer surface 24 of the device 10 comprises a colored reference strip 36 which corresponds to the color of the thermochromic material when the thermal bridge 22 reaches a predetermined temperature. The predetermined temperature is a temperature at which the medicament can be more comfortably injected into a patient. The user can compare the color of the thermochromic part of the outer surface 24 to the colored region to determine that the device is ready for use. Suitable thermochromic materials may comprise a liquid crystal material or a leuco dye.

It will be appreciated that increasing the temperature of the medicament will cause a corresponding decrease in the medicament’s viscosity. Advantageously, for a given force applied to the piston 14, decreasing the viscosity of the medicament will result in a corresponding increase in the rate at which the medicament is expelled from the device 10.

Patients may prefer that an injection occurs quickly. Therefore, the present disclosure allows medicament to be more quickly heated from its storage temperature so that a patient or user of the device 10 may inject medicament at a preferred rate.

In one embodiment, the colored reference strip 36 represents a color scale, wherein uniquely colored increments of the scale correspond to the color of the thermochromic strip 35 at different rates of injection. Each rate of injection may be labelled adjacent the corresponding increment. Alternatively, the colored reference strip 36 may be a continuous scale so that it fades from one color representing a slower rate of injection to another representing a faster rate of injection. The continuous scale may be provided with corresponding labels such as ‘fast’ and ‘slow’.

Also according to the present disclosure there is provided a system comprising a dock 40 for heating the injector device 10 described above. The dock 40 - referred to hereinafter as simply a heater 40′ - is shown in FIG. 6. The heater 40 is a standalone component of the system that is configured to heat the injector device 10 following removal of the injector device 10 from a refrigerator. The heater 40 comprises a base 41 for standing the heater on a horizontal surface and a housing 42 to enclose internal components of the heater 40. The housing 42 is provided with a U-shaped channel 43 or docking surface for receiving the device 10. The U-shaped channel 43 extends parallel to the base 41, so that the injector device 10 is stably supported when received therein. The heater 40 comprises a heater element (not shown) to heat the U-shaped channel 43. The heater element is internal to the housing 42. Any suitable electronic heating element may be used. The heater element may be activated automatically by a pressure switch when the device 10 is received in the U-shaped channel 43. Alternatively, the heater 40 may be provided with a switch 44 in the housing 42 for the user to manually activate the heater 40 after placing the injector device 10 in the U-shaped channel 43. It shall be appreciated that in other embodiments, the docking surface need not be U-shaped, but may be any shape that enables effective heat transfer between the heater 40 and the injector device 10. For example, the docking surface may be toroidal to surround the injector device 10 when the injector device 10 is received therein.

The table below gives example materials and their thermal conductivities that can be used to form the thermal bridge. All thermal conductivities are recited for the corresponding material at room temperature

| | |
|---|---|
| Material | Thermal conductivity |
| Stainless steels | 15-25 W/mK |
| Aluminium and its alloys | 75-140 W/mK |
| Carbon composite materials comprising carbon nanotubes such as CN | 1000 - 3500 W/mK |

The embodiments of injector devices described herein are configured to receive either a cartridge of medicament or a syringe pre-filled with a medicament. Herein, the term “medicament container” is intended to encompass both a cartridge of medicament and a pre-filled syringe.

The terms “drug” or “medicament” are used herein to describe one or more pharmaceutically active compounds. As described below, a drug or medicament can include at least one small or large molecule, or combinations thereof, in various types of formulations, for the treatment of one or more diseases. Exemplary pharmaceutically active compounds may include small molecules; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more of these drugs are also contemplated.

The term “drug delivery device” shall encompass any type of device or system configured to dispense a drug into a human or animal body. Without limitation, a drug delivery device may be an injector device (e.g., syringe, pen injector, auto injector, large-volume device, pump, perfusion system, or other device configured for intraocular, subcutaneous, intramuscular, or intravascular delivery), skin patch (e.g., osmotic, chemical, micro-needle), inhaler (e.g., nasal or pulmonary), implantable (e.g., coated stent, capsule), or feeding systems for the gastrointestinal tract. The presently described drugs may be particularly useful with injector devices that include a needle, e.g., a small gauge needle.

The drug or medicament may be contained in a primary package or “drug container” adapted for use with a drug delivery device. The drug container may be, e.g., a cartridge, syringe, reservoir, or other vessel configured to provide a suitable chamber for storage (e.g., short- or long-term storage) of one or more pharmaceutically active compounds. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about - 4° C. to about 4° C.). In some instances, the drug container may be or may include a dual-chamber cartridge configured to store two or more components of a drug formulation (e.g., a drug and a diluent, or two different types of drugs) separately, one in each chamber. In such instances, the two chambers of the dual-chamber cartridge may be configured to allow mixing between the two or more components of the drug or medicament prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drug delivery devices and drugs described herein can be used for the treatment and/or prophylaxis of many different types of disorders. Exemplary disorders include, e.g., diabetes mellitus or complications associated with diabetes mellitus such as diabetic retinopathy, thromboembolism disorders such as deep vein or pulmonary thromboembolism. Further exemplary disorders are acute coronary syndrome (ACS), angina, myocardial infarction, cancer, macular degeneration, inflammation, hay fever, atherosclerosis and/or rheumatoid arthritis.

Exemplary drugs for the treatment and/or prophylaxis of diabetes mellitus or complications associated with diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the term “derivative” refers to any substance which is sufficiently structurally similar to the original substance so as to have substantially similar functionality or activity (e.g., therapeutic effectiveness).

Exemplary insulin analogues are Gly(A21), Arg(B31), Arg(B32) human insulin (insulin glargine); Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Exemplary insulin derivatives are, for example, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl- ThrB29LysB30 human insulin; B29-N-(N-palmitoyl-gamma-glutamyl)-des(B30) human insulin; B29-N-(N-lithocholyl-gamma-glutamyl)-des(B30) human insulin; B29-N-(w-carboxyheptadecanoyl)-des(B30) human insulin and B29-N-(ω-carboxyhepta¬decanoyl) human insulin. Exemplary GLP-1, GLP-1 analogues and GLP-1 receptor agonists are, for example: Lixisenatide / AVE0010 / ZP10 /Lyxumia, Exenatide / Exendin-4 / Byetta / Bydureon / ITCA 650 / AC-2993 (a 39 amino acid peptide which is produced by the salivary glands of the Gila monster), Liraglutide / Victoza, Semaglutide, Taspoglutide, Syncria / Albiglutide, Dulaglutide, rExendin-4, CJC-1134-PC, PB-1023, TTP-054, Langlenatide / HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An exemplary oligonucleotide is, for example: mipomersen / Kynamro, a cholesterol-reducing antisense therapeutic for the treatment of familial hypercholesterolemia.

Exemplary DPP4 inhibitors are Vildagliptin, Sitagliptin, Denagliptin, Saxagliptin, Berberine.

Exemplary hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Exemplary polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 / Synvisc, a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region.

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Exemplary antibodies are anti PCSK-9 mAb (e.g., Alirocumab), anti IL-6 mAb (e.g., Sarilumab), and anti IL-4 mAb (e.g., Dupilumab).

The compounds described herein may be used in pharmaceutical formulations comprising (a) the compound(s) or pharmaceutically acceptable salts thereof, and (b) a pharmaceutically acceptable carrier. The compounds may also be used in pharmaceutical formulations that include one or more other active pharmaceutical ingredients or in pharmaceutical formulations in which the present compound or a pharmaceutically acceptable salt thereof is the only active ingredient. Accordingly, the pharmaceutical formulations of the present disclosure encompass any formulation made by admixing a compound described herein and a pharmaceutically acceptable carrier.

Pharmaceutically acceptable salts of any drug described herein are also contemplated for use in drug delivery devices. Pharmaceutically acceptable salts are for example acid addition salts and basic salts. Acid addition salts are e.g. HCl or HBr salts. Basic salts are e.g. salts having a cation selected from an alkali or alkaline earth metal, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are known to those of skill in the arts.

Pharmaceutically acceptable solvates are for example hydrates or alkanolates such as methanolates or ethanolates.

Those of skill in the art will understand that modifications (additions and/or removals) of various components of the substances, formulations, apparatuses, methods, systems and embodiments described herein may be made without departing from the full scope and spirit of the present disclosure, which encompass such modifications and any and all equivalents thereof.

1-15. (canceled)

16. A drug delivery device comprising:
an outer surface;
a region for receiving a medicament container, the region having an inner surface to contact and support a medicament container received therein; and
a thermal bridge extending between the inner surface and the outer surface to conduct heat between the inner surface and the outer surface, wherein the thermal bridge has a thermal conductivity of at least 10W/mk.

17. The drug delivery device of claim 16, wherein the inner surface and the outer surface are integrally formed with the thermal bridge to provide an unbroken conductive pathway between the inner surface and the outer surface.

18. The drug delivery device of claim 17, wherein the outer surface is an outer surface of a housing.

19. The drug delivery device of claim 18, wherein the thermal bridge is integrally formed with the housing.

20. The drug delivery device of claim 19, wherein the thermal bridge comprises a region of the housing having increased wall thickness so that an internal diameter of the region of the housing having increased wall thickness is substantially equal to an external diameter of a medicament container to be received therein.

21. The drug delivery device of claim 18, wherein the housing comprises a first part and a second part, and wherein the thermal bridge is integrally formed in the first part.

22. The drug delivery device of claim 21, wherein the thermal bridge comprises a collar disposed between the first part and the second part of the housing, wherein the collar comprises a cylindrical section with an internal diameter substantially equal to an external diameter of a medicament container to be received therein.

23. The drug delivery device of claim 16, wherein the thermal bridge comprises a material having a coefficient of thermal conductivity greater than 70W/mK.

24. The drug delivery device of claim 18, wherein the thermal bridge is configured to hold a medicament container in a fixed position relative to the housing by interference fit.

25. The drug delivery device of claim 18, comprising a medicament container.

26. The drug delivery device of claim 25, wherein the thermal bridge holds the medicament container in a fixed position relative to the housing, the thermal bridge being bonded to the medicament container by an adhesive having a thermal conductivity greater than 2W/mK.

27. The drug delivery device of claim 25, wherein the medicament container comprises a material having a coefficient of thermal conductivity greater than 10W/mK.

28. The drug delivery device of claim 16, comprising a thermochromic material configured to change color in response to a change in a temperature of the thermal bridge.

29. The drug delivery device of claim 28, wherein the outer surface comprises a colored region which corresponds to a color of the thermochromic material when the thermal bridge reaches an injection temperature.

30. A system comprising:
a heater comprising a heated docking surface; and
a drug delivery device comprising an outer surface, an inner surface, and a thermal bridge extending between the inner surface and the outer surface,
wherein the heated docking surface is configured to contact the outer surface of the drug delivery device when the drug delivery device is placed on the heated docking surface.

31. A method of operating a drug delivery device, the method comprising: placing an outer surface of a drug delivery device in contact with a heat source to heat a medicament within a medicament container of the drug delivery device from a storage temperature to an injection temperature, the drug delivery device comprising a thermal bridge extending between the medicament container and the outer surface, the thermal bridge having a thermal conductivity equal to or greater than 10W/mk.
